(19) United States
(12) Patent Application Publication
(10) Pub. No.: US 2020/0237855 A1
(43) Pub. Date: Jul. 30, 2020

(54) TREATMENT OF PTSD

(71) Applicant: Edward M. Lichten, Birmingham, MI (US)

(72) Inventor: Edward M. Lichten, Birmingham, MI (US)

(21) Appl. No.: 16/777,197

(22) Filed: Jan. 30, 2020

Related U.S. Application Data
(60) Provisional application No. 62/798,665, filed on Jan. 30, 2019.

(51) Int. Cl. A61K 38/095; A61K 31/554; A61K 38/06; A61K 35/00

(52) U.S. Cl. CPC A61K 38/095; A61K 31/554; A61K 9/0056; A61K 35/00; A61K 38/06

(57) ABSTRACT

A method of treating PTSD combines oxytocin with an additional neurotransmitter and, optionally, a minor amount of an amino acid amastatin. The composition may be deployed as either a lozenge or as a topical cream. Also, an effective legal quantity of CBD oil may be used conjointly therewith, whether directly incorporated in the composition or used separately.

### CROSS-REFERENCE TO RELATED APPLICATION

This application is a Completion Application of Co-Pending U.S. Provisional Patent Application, Ser. No. 62/798,665, filed on Jan. 30, 2019 for “Treatment of PTSD,” the disclosure of which is hereby incorporated by reference in its entirety.

### BACKGROUND OF THE INVENTION

### 1. Field of the Invention

The present invention concerns the treatment of post-traumatic shock disorder (PTSD). More particularly, the present invention concerns compositions for the treatment of PTSD. Even more particularly, the present invention concerns oxytocin-based compositions for the treatment of PTSD.

### 2. Prior Art

As is known to those skilled in the art to which the invention pertains oxytocin is a naturally produced hormone released from the hypothalamus in the mammalian brain. Oxytocin is an essential hormone for the reproduction function in females. It is released to increase uterine contractions for delivery of an unborn child and for milk letdown postpartum for breast feeding of off-spring. Ordinarily, oxytocin is released at the time of orgasm. Non-pregnancy research has found that oxytocin may have a role in the bonding between two mammals.

Oxytocin also acts as a neurotransmitter by increasing the connectivity between the various parts of the brain. Yet, as is well known, stress reduces oxytocin release. Because oxytocin does not cross paths through the blood-brain barrier, nasal oxytocin has been used to break down the blood-brain barrier, but with inconsistent results. Serum and blood measurements only show the peripheral levels of the hormone.

Also, oxytocin is well absorbed from the vault of the vagina and from the rectum through the lymphatic system. It is also believed that oxytocin's effect may be increased by its acting in conjunction with other neurotransmitters, as well as with an over-the-counter amino acid, such as an amastatin.

Based upon these beliefs, it is now postulated that the benefits or the effects of oxytocin can have an advantageous effect on the treatment of PTSD when used either alone or in combination with other compounds.

Thus, the present invention is directed to the use of oxytocin in the treatment of PTSD.

### SUMMARY OF THE INVENTION

In accordance with the present invention, the is provided a topical application, preparation or a lozenge for utilization in connection with the treatment of PTSD based upon oxytocin.

Preferably, the oxytocin is used in combination with additional components such as an additional or second neurotransmitter and, optionally, an amastatin.

An effective amount of CBD, all within the legal limits, may also be used in admixture therewith within the legal limits.

For a more complete understanding of the present invention, reference is made to the following description of the invention.

### DESCRIPTION OF THE INVENTION

In accordance with the present invention there is provided a lozenge or topical application preparation consisting essentially of oxytocin in either natural or synthetic association with an additional neurotransmitter and, optionally, an amastatin.

Thus, and in a first aspect the present invention, generally, comprises a lozenge composition comprising the oxytocin, at least one additional neurotransmitter and the amino acid-based amastatin. The composition, is, preferably, admixed with a lozenge composition comprising a source of deionized water, sugar, and other neutral or non-active components.

Thereafter, the admixture is heated to form a homogeneous solution which is then hardened by baking at a suitable temperature to drive off the water in the suspension to form a lozenge.

The lozenge can be orally ingested or can be inserted into the vagina or inserted into the rectum where it is readily absorbed.

As a lozenge typically, the preparation is administered on a daily basis in dosages ranging from about 25 mg to about 400 mg. Preferably, each lozenge weighs about 100 mg.

Typically, when used as a lozenge, the lozenge, generally, comprises from about 50 IUs to 150 IUs of oxytocin and, preferably, from about 75 to about 125 IUs of oxytocin, based upon the entire weight of the lozenge.

The lozenge further comprises an additional neurotransmitter and, where used, an effective amount of an amastatin in admixture with the oxytocin.

Oxytocin, commercially, is available as a powder which can be put into aqueous suspension. Such a suspension is used to prepare the lozenge.

Such suspension will, ordinarily, comprise from about 1.25 to about 1.5 cm³ of oxytocin, by volume, based on the entire volume of the suspension.

The additional neurotransmitter is present in an amount ranging from about 1% to about 10%, by weight, based upon the entire weight of the lozenge.

Among the useful neurotransmitters are dopamine, serotonin, seroquel and the like, as well as mixtures thereof.

The second or additional neurotransmitter is present in the lozenge in an amount ranging from about 1 mg to about 10 mg based on a 100 mg, based upon 100 mg, by weight, of the lozenge. Preferably, the additional neurotransmitter is seroquel.

The amastatins contemplated for use herein are amino acids such as alanine, glutamic acid, lucene, essential proteinogenics and the like as well as mixtures thereof.

As a topical compound, the active ingredients can be introduced into a deliverable such as a body lotion, skin cream and the like. Preferably, a skin cream is used.

Typically, the active ingredients can be admixed with skin cream, such as Lubriderm® or the like in the above-noted amounts by first preparing the oxytocin aqueous suspension and then admixing it with the lozenge non-active ingredients and the other components at room temperature with agitation.

It should be noted that it is possible to incorporate into the composition directly or to ingest concurrently with the lozenge and apart therefrom a suitable quantity of CBD oil.

Alternatively, the CBD oil may be admixed in its crystalline form with the other ingredients in the lozenge composition and form part of the lozenge, or it can be deposited on the lozenge surface after it is hardened.

Alternatively, the CBD oil can be embedded into a tablet.

Similarly, the CBD oil may be in admixture with the topical composition.

With or without the CBD oil incorporated into it, the lozenge may be used conjointly with other pharmaceuticals, such as with well-known anti-anxiety compounds, for example, 5-hydroxytrytophan; melatonin, serotonin and the like.

Where CBD oil is used, it is used in legally accepted limits, based upon having no more than 0.3% THC.

Having, thus, described the invention, what is claimed is:

1. A method for treating PTSD comprising:
administering an effective amount of a preparation consisting essentially an admixture of oxytocin and an additional neurotransmitter.

2. The method of claim 1 wherein the preparation is administered as a lozenge.

3. The method of claim 2 wherein the lozenge generally comprises:
(a) from about 50 IUs to about 150 IUs of oxytocin; b) from about 1% to about 10%, by weight, of the additional neurotransmitter based upon the entire weight of the lozenge.

4. The method of claim 1 wherein the additional neurotransmitter is selected from the group consisting of dopamine, serotonin, seroquel and mixtures thereof.

5. The method of claim 4 wherein the additional neurotransmitter is seroquel.

6. The method of claim 1 wherein the prepartion further includes a minor amount of an amino acid amastatin selected from the group consisting of alanine, glutamic acid, lucine, essential proteinogenics and mixtures thereof.

7. The method of claim 1 which further includes an effective amount of CBD oil in the admixture.

8. The method of claim 1 wherein the oxytocin is in an aqueous suspension and wherein the suspension comprises from about 1.25 to about 1.5 cubic centimeters of oxytocin by volume, based upon the entire volume of the suspension.

9. The method of claim 1 wherein the preparation is a topical compound comprising in admixture:
(a) a skin cream;
(b) the oxytocin; and
(c) the additional neurotransmitter.

10. The method of claim 9 wherein the topical compound generally comprises:
(a) from about 50 Us to about 150 IUs of oxytocin;
(b) b) from about 1% to about 10% by weight of the additional neurotransmitter based upon the entire weight of the preparation.

11. The method of claim 9 wherein the neurotransmitter is selected from the group consisting of dopamine, serotonin, seroquel and mixtures thereof.

12. The method of claim 9 which further includes a minor amount of an amino acid amastatin selected from the group consisting of alanine, glutamic acid, lucine, essential proteinogenics and mixtures thereof.

13. The method of claim 9 wherein the preparation further includes an effective amount of CBD oil in the admixture.

14. The method of claim 9 wherein the oxytocin is deployed in an aqueous suspension, wherein the suspension comprises from about 1.25 to about 1.5 cubic centimeters of oxytocin, by volume, based upon the entire volume of the suspension.
